(19) United States
(12) Patent Application Publication
(10) Pub. No.: US 2017/0116665 A1
(43) Pub. Date: Apr. 27, 2017

(54) SMART ORAL HYGIENE AID

(71) Applicant: Abdulrahman Obaid ALZAHRANI, Corvallis, OR (US)

(72) Inventor: Abdulrahman Obaid ALZAHRANI, Corvallis, OR (US)

(21) Appl. No.: 14/921,620

(22) Filed: Oct. 23, 2015

(51) Int. Cl. G06Q 30/06; A46B 11/00; A46B 5/00; G08B 21/18; A61C 17/22; A61C 17/34; A46B 15/00; A61B 5/00; A46B 9/04; A46B 17/06

(52) U.S. Cl. CPC G06Q 30/0641; A46B 9/04; A46B 11/0072; A46B 11/0006; A46B 5/0025; A46B 17/065; A61C 17/221; A61C 17/3481; A46B 15/0042; A61B 5/4547; G08B 21/182

(57) ABSTRACT

A method, system, apparatus and non-transitory computer readable medium for determining a user's state of oral hygiene including a hollow handle member having an internal chamber to house a fluid cartridge, a head member connected on one end to the handle member to rotate at fixed angular increments, a head platform including at least one bristle platform, an fluid conduit that is disposed within the head member and a fluid cartridge including at least one fluid chamber and a negotiating neck to attach to the negotiating portal, and circuitry configured to detect a brushing motion, identify at least one type of the at least one oral hygiene fluid within the oral hygiene fluid chamber and regulate a predetermined output quantity of the oral hygiene fluid to the moldable shape head via the oral hygiene fluid conduit based on the detected brushing motion.

### FIELD OF THE INVENTION

The present disclosure relates generally to systems and methods for monitoring and improving oral hygiene including devices and methods to help a user maximize effectivness of oral hygiene applciations.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Studies indicate that many people, especially children, do not properly employ commonly used hygiene devices, such as toothbrushes and dentifrices. To overcome this problem, some have employed musical entertainment devices and timers to motivate users to comply with well-established protocols. However, the effectiveness of such devices has not been adequately documented to yield improved results in oral hygiene. Therefore, there exists a need for a multi-solution enabling hygiene device that can also monitor progress of oral hygiene to enable users to maximize effectiveness of oral hygiene applications.

### SUMMARY

Systems, methods and apparatus for maintaining and monitoring oral health and hygiene that include a hollow handle member having an internal chamber to house an oral hygiene fluid cartridge, a head member hingedly connected on one end to the handle member and configured to rotate at fixed angular increments within a range of 0 to 180 degrees between the head member and the handle member, a head platform secured to an end of the head member opposite from the handle member including at least one bristle platform; an oral hygiene fluid conduit that is disposed within the head member and connected on one end to the head platform and connected on another end other end to a fluid negotiating portal within the internal chamber of the hollow handle member; an oral hygiene fluid cartridge including at least one oral hygiene fluid chamber and a negotiating neck operatively configured to attach to the negotiating portal, and circuitry to detect a brushing motion, identify at least one type of the at least one oral hygiene fluid within the oral hygiene fluid chamber and identify the quantity of the at least one type of the at least one oral hygiene fluid, and regulate a predetermined output quantity of the oral hygiene fluid to the moldable shape head via the oral hygiene fluid conduit based on the detected brushing motion. Within the oral hygiene system, the head platform may be an elastically deformable head platform including at least two bristle platforms that are adapted to change disposition with regards to one another within the elastically deformable head platform. In one embodiment, each of the at least two bristle platforms includes at least one irrigation nozzle operatively connected to the oral hygiene fluid conduit, wherein the irrigation nozzle is designed to output oral hygiene fluid received from the oral hygiene fluid conduit onto a bristle surface of each of the at least two bristle platforms. Furthermore, the irrigation nozzle may be a bristle within each of the at least two bristle platforms and the elastically deformable head platform dimensions are modifiable in longitudinal and latitudinal directions.

In some exemplary aspects, the oral hygiene fluid cartridge includes one or more oral hygiene fluid chambers and at least one of the chambers contains fluid used to clean the oral hygiene fluid conduit of any residue resultant from prior uses. Furthermore, the one or more oral hygiene fluid chambers may be either vacuum sealed or pressure sealed. Additionally, the one or more oral hygiene fluid chambers are vacuum sealed such that when the oral hygiene fluid cartridge is connected to the fluid negotiating portal, the fluid negotiating portal further comprising a fluid regulator configured to negotiate fluid flow from the vacuum sealed oral hygiene fluid chambers into the oral hygiene fluid conduit.

The oral hygiene system includes the oral hygiene fluid cartridge that further includes a near field communication (NFC) tag that is programmed to automatically identify the type and content of the hygiene fluid cartridge when the hygiene fluid cartridge is within the oral hygiene system. The oral hygiene fluid maybe toothpaste or different types of toothpaste that are housed within different oral hygiene fluid chambers. The different types of toothpaste may include one or more combination of different flavored toothpaste, different texture tooth paste, mouth wash, oral hygiene fluid conduit cleaner, and tooth whitener.

The oral hygiene system may include circuitry such as memory configured to store usage preferences and prior usage history, wherein the circuitry is further configured to regulate an output quantity from different oral hygiene fluid chambers based on stored usage preferences or prior usage history. The circuitry is further configured to measure a quality of dental hygiene and automatically regulate an output of oral hygiene fluid from at least one chamber of the oral hygiene fluid cartridge based on the measured quality of dental hygiene. The measured quality of dental hygiene is related to plaque buildup.

In some exemplary aspects, a method of monitoring and maintaining dental hygiene is presented and includes automatically detecting brushing activity using motion detection circuitry, determining toothpaste quantity based on tracked average history and detected motion, detecting toothpaste levels in a toothpaste cartridge, outputting warning to change toothpaste cartridge when levels falls below a threshold, and displaying toothpaste cartridge order options. The method may further include measuring a quality of dental hygiene and displaying a toothpaste solution based on the measured quality of dental hygiene. Additionally the method may further include irrigating an oral hygiene fluid conduit with oral hygiene fluid from an oral hygiene fluid cartridge in response to a detected brushing activity; and irrigating the oral hygiene fluid conduit with cleaning fluid from the oral hygiene fluid cartridge after the detected brushing activity ends. Other aspects of the present disclosures include a non-transitory computer readable medium having computer-readable instructions thereon which when executed by a computer cause the computer to perform a method according to the method described above.

The foregoing paragraphs have been provided by way of general introduction, and are not intended to limit the scope of the following claims. The described aspects of the present disclosures, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a system overview of an oral hygiene monitoring system and method configuration according to an exemplary aspects of the present disclosure;

FIG. 2 describes a system sensor configuration within oral hygiene monitoring system according to exemplary aspects of the present disclosure;

FIG. 3A is a system overview of oral hygiene monitoring system including a display and cartridge holding assembly according to exemplary aspects of the present disclosure;

FIG. 3B is a system overview of oral hygiene monitoring device including a movable head assembly according to exemplary aspects of the present disclosure;

FIG. 4 describes a side view of oral hygiene monitoring device including internal structure according to exemplary aspects of the present disclosure;

FIG. 5 describes a side view of oral hygiene monitoring device including internal components according to exemplary aspects of the present disclosure;

FIG. 6 describes a view of head assembly including flexible a deformable head platform including multiple movable bristle platforms according to exemplary aspects of the present disclosure;

FIG. 7 describes a sample cartridge assembly with internal components and interfaces according to an exemplary aspects of the present disclosure;

FIG. 8 describes a sample cartridge assembly with possible internal components according to an exemplary aspects of the present disclosure;

FIG. 9 describes a flow diagram illustration for detecting brushing action and dispensing oral hygiene solution according to an exemplary aspects of the present disclosure;

FIG. 10 describes a flow diagram illustration for measuring, storing and presenting oral hygiene habits according to an exemplary aspects of the present disclosure;

FIG. 11 describes a sample list of measured and stored metrics associated with oral hygiene activities according to an exemplary aspects of the present disclosure;

FIG. 12 describes a sample interchangeable head configuration to output high pressure liquid or steam according to exemplary aspects of the present disclosure;

FIG. 13 describes a sample interchangeable head assembly for aiding in tongue cleaning according to exemplary aspects of the present disclosure; and

FIG. 14 describes a radiation head configurations for scanning status of oral hygiene according to exemplary aspects of the present disclosure.

### DETAILED DESCRIPTION

The detailed description set forth below in connection with the appended drawings is intended as a description of presently preferred aspects of the disclosure and is not intended to represent the only forms in which the present disclosure may be constructed and or utilized.

For purposes of illustration, programs and other executable program components are illustrated herein as discrete blocks, although it is recognized that such programs and components may reside at various times in different storage components and are executed by the data processor(s) of the computers. Further, each block within a flow chart may represent method function(s), operation(s), or act(s) and one or more elements for performing the method function(s), operation(s), or act(s). In addition, depending upon the implementation, the corresponding one or more elements may be configured in hardware, software, firmware, or combinations thereof.

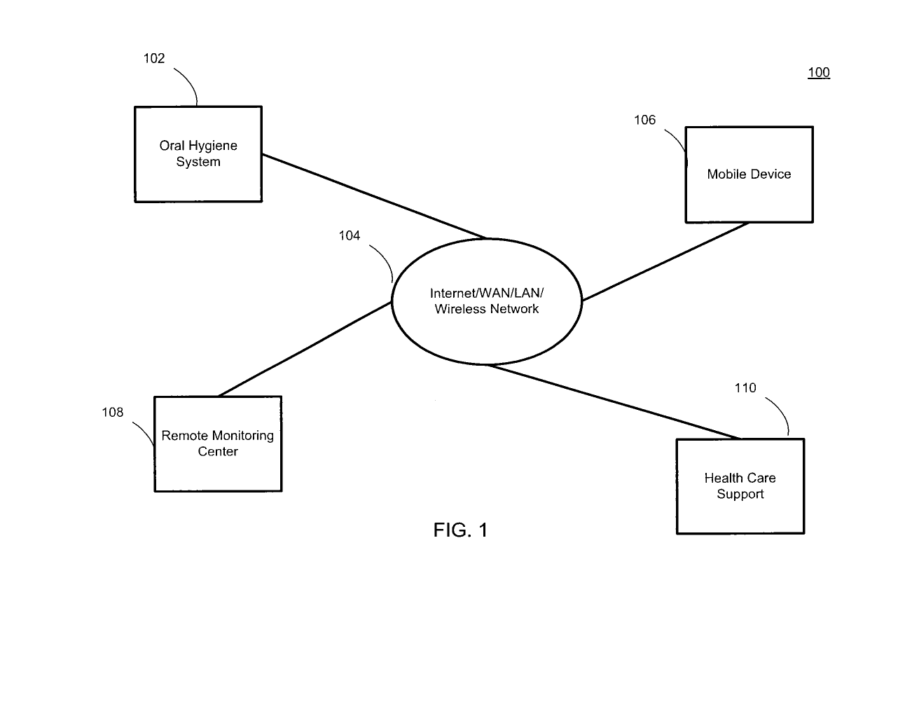

FIG. 1 is a system overview of an oral hygiene monitoring system and method configuration according to exemplary aspects of the present disclosure. Oral hygiene monitoring system 100 includes oral hygiene system 102, internet 104, mobile device 106, remote monitoring center 106 and health care support system 110. Oral hygiene system 102 may be a hand held device with multiple application platforms for conducting oral hygiene operations, such cleaning mouth, brushing teeth, teeth whitening, treating sensitive teeth, hygiene detection and monitor a user's behavior for further analysis. Oral hygiene system 102 may be able to connect to other devices, such as mobile device 106, remote monitoring center 108 and health care support 110 through a network connection 104. Network connection 104 may be an internet connection, a wide area network (WAN) connection, a local area network (LAN) connection, a Bluetooth connection, or any other means of wireless connectivity. Mobile device 106 may be any type of smart mobile device with ability to connect to and control other devices through wireless connectivity such as WiFi, cellular, or Bluetooth connectivity.

Oral hygiene system 102 may convey certain measured information to the mobile device 106 through network connection 104. Such information may include cartridge content levels, frequency of most used supplies, number of active sessions, session length, tooth brush pressure, user goals and user goals vs logged data, dental hygiene progress, ordered supplies and status and dr. notifications, instructions and appointments as one of ordinary skill in the art may appreciate. The mobile device may store such information, further manipulate such information, present the information for the user in a larger viewing capacity for the user, or further communicate the information to a third party, such as remote monitoring center 108 or health care support 110. Remote monitoring center can be dedicated center for monitoring oral health or a third person designated as a person who has an interest in continuously measuring oral health information of the user. For example, remote monitoring center 108 can be a mobile device of a friend, wife, mom, family member, etc. If the person is disabled, for example, the remote monitoring center may be a nurse's station, a care taker's mobile device (or alternatively, another medical bracelet) whose has an interest to continuously monitor a person's oral hygiene information.

In cases where oral hygiene monitoring system detects a threshold excess violation, oral hygiene system 102 contacts health care support 110. Health care support 110 can include a doctor's office, a designated preferred medical practitioner or an emergency center that can dispatch immediate assistance.

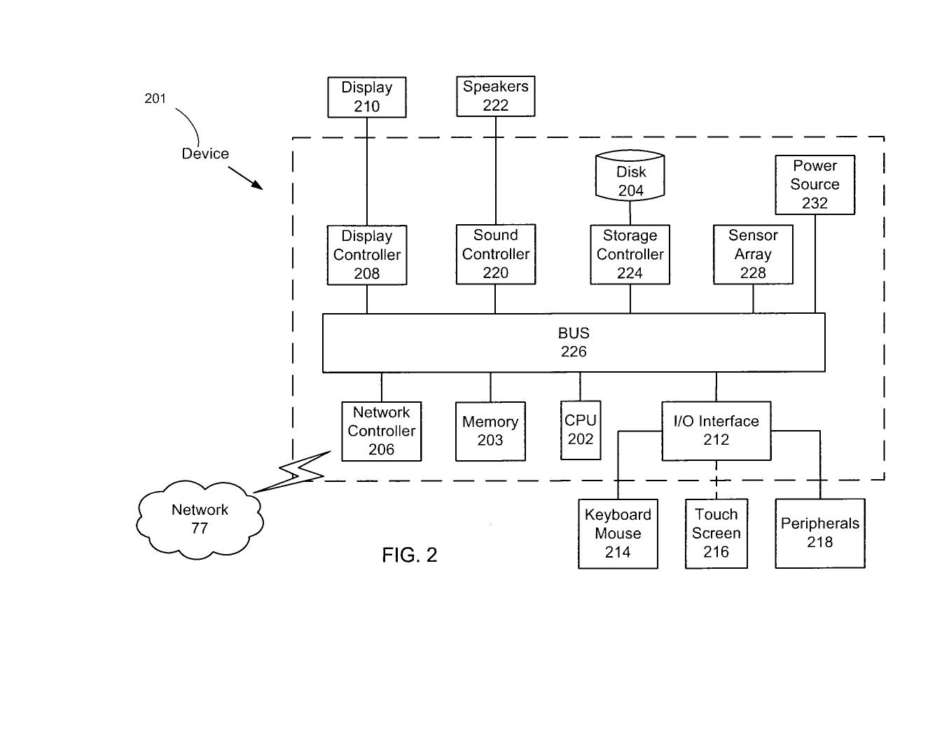

FIG. 2 is describes a system sensor configuration within oral hygiene system 200 according to exemplary aspects of the present disclosure. Oral hygiene system 200 includes device 201 (such as an electronic toothbrush serving as a modular platform for oral hygiene monitoring sensors) that includes a CPU 202 which performs control and processing calculations further discussed hereinafter. The process data and instructions may be stored in memory 203. These processes and instructions may also be stored on a storage medium disk 204 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device communicates, such as a server or computer. The memory 203 may store application program instructions and store default and updated user preferences. In aspects of the present disclosure, the memory 203 may store application programs used to implement aspects of the functionality of the oral hygiene monitoring system described herein. The memory 203 may also store raw data, recorded data, and/or calculated data, downloaded data and/or as explained in further detail below, the memory 203 may act as a data storage buffer. The memory 203 may include both read only memory and random access memory, and may further include memory cards or other removable storage devices. The memory 203 may store data in memory locations of predetermined size, i.e., bytes, words, sectors, and/or blocks, such that only a certain quantity of data may be saved for a particular application of the CPU 202.

Further, the present advancements may be provided as a utility application or component of an operating system, or combination thereof, executing in conjunction with CPU 202 and an operating system such as Microsoft Windows 7, UNIX, Solaris, LINUX, Apple MAC-OS and other systems known to those skilled in the art.

CPU 202 may be a Xenon or Core processor from Intel of America or an Opteron processor from AMD of America, or may be other processor types that would be recognized by one of ordinary skill in the art. Alternatively, the CPU 202 may be implemented on an FPGA, ASIC, PLD or using discrete logic circuits, as one of ordinary skill in the art would recognize. Further, CPU 202 may be implemented as multiple processors cooperatively working in parallel to perform the instructions of the inventive processes described above.

Device 201 also includes a network controller 206, such as an Intel Ethernet PRO network interface card from Intel Corporation of America, for interfacing with network 77.

Network controller 206 enables CPU 202 to wirelessly communicate with other components of the oral hygiene monitoring system and or other mobile and monitoring systems such as mobile devices, emergency services and medical care services, such as those described in further detail below. CPU 202 may communicate with other components over a personal area network or local area network using, for example, one or more of the following protocols: ANT, ANT+ by Dynastream Innovations, Bluetooth, Bluetooth Low Energy Technology, BlueRobin, or suitable wireless personal or local area network protocols. Other known communication protocols suitable for an oral hygiene system may also be used. In one exemplary aspect, network controller 206 is a low-power controller and may be a two-way communication controller 206, or a one-way transmitter or a one-way receiver.

As can be appreciated, the network 77 can be a public network, such as the Internet, or a private network such as an LAN or WAN network, or any combination thereof and can also include PSTN or ISDN sub-networks. The network 77 can also be wired, such as an Ethernet network, or can be wireless such as a cellular network including EDGE, 3G and 4G wireless cellular systems. The wireless network can also be Wi-Fi, Bluetooth, or any other wireless form of communication that is known.

The device 201 further includes a display controller 208, such as a NVIDIA GeForce GTX or Quadro graphics adaptor from NVIDIA Corporation of America for interfacing with display 210, such as a Hewlett Packard HPL2445w LCD monitor. A general purpose I/O interface 212 interfaces with a keyboard and/or mouse 214 as well as a touch screen panel 216 on or separate from display 210. General purpose I/O interface also connects to a variety of peripherals 218 including printers and scanners, such as an OfficeJet or DeskJet from Hewlett Packard. General purpose I/O interface 212 may enable a user to set user goals regarding level of health and quality of oral hygiene practices as well as user preferences in terms of products and oral hygiene solution used, modes of operation and other input needs as one of ordinary skill in the art may appreciate.

A sound controller 220 is also provided in device 201, such as Sound Blaster X-Fi Titanium from Creative, to interface with speakers/microphone 222 thereby providing sounds and/or music. Sound controller 220 allows a user to control alarm signal sounds designed to alert the user to a predefined condition. Sound controller 220 may further be used to output, via speakers 222 oral hygiene instructions, guided instructions related to content of the oral hygiene cartridge, or instructions on how to mix different content of the oral hygiene cartridge as will be further discussed hereinafter.

The general purpose storage controller 224 connects the storage medium disk 204 with communication bus 226, which may be an ISA, EISA, VESA, PCI, or similar, for interconnecting all of the components of the device. A description of the general features and functionality of the display 210, keyboard and/or mouse 214, as well as the display controller 208, storage controller 224, network controller 206, sound controller 220, and general purpose I/O interface 212 is omitted herein for brevity as these features are known. An array of sensors may be incorporated within device 201, such as sensor array 228.

Device 201 may be powered by power source 232. The power source 232 provides power to all elements of device 201. The power source 232 may be a battery. The power source may also be built into the CPU 202 or removable from the CPU 202, and may be rechargeable or non-rechargeable. The power source 232 may be recharged by a cable attached to a charging source, such as a universal serial bus (“USB”), FireWire, Ethernet, Thunderbolt, headphone cable, or a specially designed cable attached to a personal computer. The power source 232 may also be recharged by inductive charging, which uses an electromagnetic field to transfer energy from an inductive charger to the power source 232 when the two are brought in close proximity. Thus, the power source need not be plugged into one another via a cable. A docking station may also be used to facilitate charging.

Device 201 may also include a timer (not shown). The timer may be a clock that tracks absolute time and/or determines elapsed time. In some exemplary aspects, the timer may be used to timestamp certain data records, such that the time that certain data was measured or recorded may be determined and various timestamps of various pieces of data may be correlated with one another.

Device 201 may further include a data port as part of peripherals 218. The data port facilitates information transfer to and from the CPU 202 and may be, for example, a USB port. The data port can additionally or alternatively facilitate power transfer to power source 232, in order to charge power source 232.

Sensor array 228 may include an acceleration sensor (not shown). The acceleration sensor measures the acceleration of the CPU 202 or device 201 when it is moved or used by an individual. The acceleration sensor may measure the acceleration due to the earth's gravitational field. In one exemplary aspect, the acceleration sensor may include a tri-axial accelerometer that measures acceleration in three orthogonal directions. Of course two, three, or more separate accelerometers may be used in the alternative without departing from the scope of the present disclosure. The acceleration sensor is used to determine when device 201 is used, such as to activate dispensing of oral hygiene solution from oral hygiene cartridge as will be further discussed herein.

Sensor array 228 may further include a magnetic field sensor that measures the strength and direction of magnetic fields in the vicinity of the device 201. Accordingly, when the device 201 is physically coupled to an individual, the magnetic field sensor may measure the strength and direction of magnetic fields in the vicinity of the individual, including the earth's magnetic field. In one exemplary aspect, the magnetic field sensor may be a vector magnetometer. The magnetic field sensor may also be a tri-axial magnetometer that measures the magnitude and direction of a resultant magnetic vector for the total local magnetic field in three dimensions. Two, three, or more separate magnetometers may be used as one of ordinary skill would recognize. In similar fashion, the magnetic field sensor may be used to determine when device 201 is being used by a user.

In one example, the acceleration sensor and the magnetic field sensor may be contained within a single accelerometer-magnetometer circuit integrated circuity such as LSM303DLHC made by STMicroelectronics of Geneva, Switzerland.

Sensor array 228 may further include an angular momentum sensor (not shown), which may be a gyroscope, and is adapted to measure the angular momentum or orientation of the device 201. Accordingly, when the device 201 is physically coupled to an individual, the angular momentum sensor may measure the angular momentum or orientation of the individual. The angular momentum sensor may be a tri-axial gyroscope that measures angular rotation about three orthogonal axes. Two, three, or more separate gyroscopes may be used instead, however. In an exemplary aspect of the present disclosure, the angular momentum sensor may be used to calibrate measurements made by one or more of the acceleration sensor and the magnetic field sensor.

The angular momentum sensor, acceleration sensor or magnetic field sensor or any combination thereof may be combined to form motion sensing circuitry that detects brushing motion of a user and will be referred to hereinafter as motion sensing circuitry or motion sensor.

Sensor array 228 further includes a temperature sensor for measuring body temperature and/or ambient temperature. The temperature sensor may be, for example, a thermometer, a thermistor, or a thermocouple that measures changes in the temperature. The temperature sensor may also be non-invasive laser temperature sensor housed within a head member of the oral hygiene device 201 to measure the temperature of a user and notify the user of any potential risks while the user is brushing his teeth for example.

Sensor array 282 further includes a position/GPS sensor that may be an electronic satellite position receiver determines its location (i.e., longitude, latitude, and altitude) using time signals transmitted along a line-of-sight by radio from satellite position system satellites. Known satellite position systems include the GPS system, the Galileo system, the BeiDou system, and the GLONASS system. The position/GPS sensor may also be an antenna that communicates with local or remote base stations or radio transmission transceivers to determine the location of device 201 using radio signal triangulation or other similar principles. The position/GPS sensor data may allow CPU 202 to detect information that may be used to measure and/or calculate position waypoints, time, location, distance traveled, speed, pace, or altitude as would be recognized by one of ordinary skill.

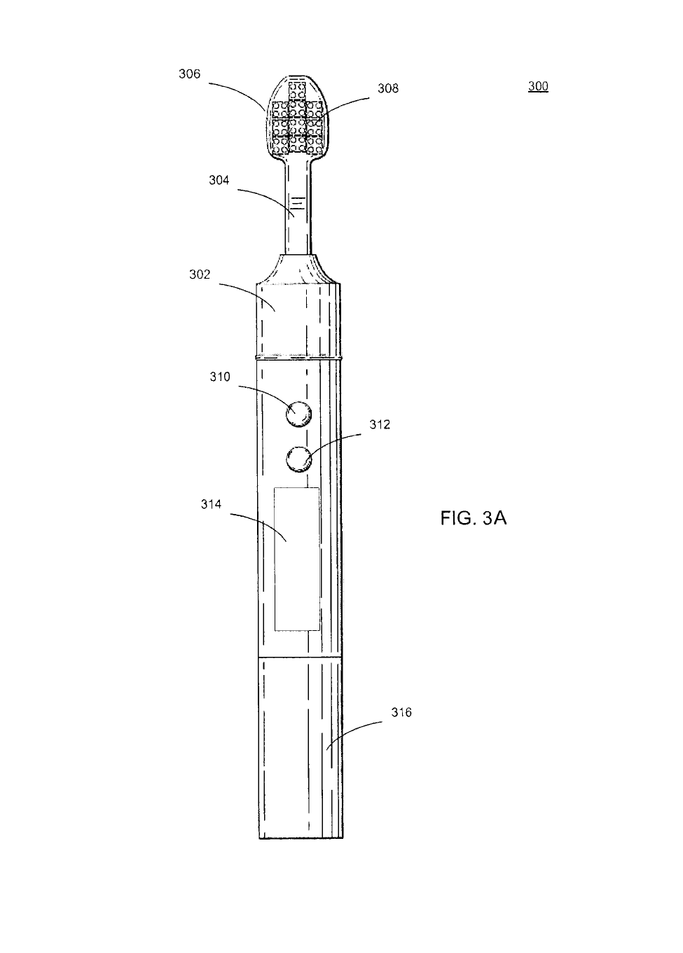

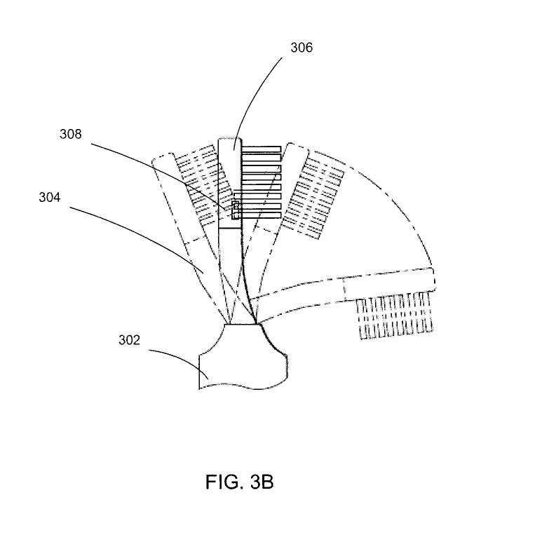

FIG. 3A is a system overview of oral hygiene monitoring system 300 including a display and cartridge holding assembly according to exemplary aspects of the present disclosure. Oral hygiene monitoring device 300 includes a hollow handle member 302, head member 304, head platform 306, bristle platforms 308, user interface components 310, 312 and 314, and sensor housing member 316. Hollow handle member 302 is a hollow member that is constructed to house a plurality of elements that make oral hygiene device 300 operate up to the desired specification. Hollow handle member also functions as the main handle for a user to hold oral hygiene device 300 and can house sensors and cartridges as will be further described herein.

Hollow handle member 302 connects on one side to head member 304 and on the other side to a sensor housing member 316, which can also be part of hollow handle member 302. Head member 304 can connect to handle member 302 in a variety of ways. In one aspects of the present disclosure head member 304 can be fixed to hollow handle member 302. Head member 304 may be connected to hollow handle member via a hinge such that head member 304 can move about an axis with predetermined degrees of freedom between head member 304 and hollow handle member 302. The aim is to allow the head member 304 to have a degree of flexibility so that it enables oral hygiene device 300 to have greater reach and application. For example, some hard to reach spots in a user's mouth may be hard to reach if the angle between head member 304 and hollow handle member 302 is 180 degrees. As such, tilting head member 304 in a direction that either increases or decreases the angle between head member 304 and hollow handle member 302 may give the user greater access to areas not covered before.

FIG. 3B is an illustration of the head movement with regards to the body according to exemplary aspects of the disclosure. Head member 304 may be connected to hollow handle member 302 via a hinge. Alternatively, head member 304 may also be connected to hollow handle member 302 via a ball bearing assembly such that the head member 304 can rotate about in multiple directions to enhance the feel and reach of oral hygiene device 300.

Head member 304 also includes head platform 306 and bristle platforms 308 described further herein. Head platform 306 includes a multitude of bristle platforms 308. Each platform can be configured to host a number of bristles that can vary in length and quantity. Head platform 306 can be made of malleable material that can change shape according to a user applied physical input. For example, and for purposes of illustration, head platform 306 may be an elastically deformable head platform that can receive a user physical input and take a shape in accordance with the applied force of the user. For example, force applied by a user may cause the bristle platforms 308 to be rearranged according to the applied force as will be further described herein.

Referring back to FIG. 3A, oral hygiene monitoring device 300 includes a plurality of user interface components. In one example, oral hygiene monitoring device 300 may include an ON/OFF button 310, a dispensing button 312 and display 314. ON/OFF button 310 may be used for when a user determines to use electronic brushing mode so to create vibrations that enable more effective cleaning. Dispensing button 312 enables a user to control the timing and amount of dispensing of an oral hygiene solution through head platform 306 as will be further described herein. In one embodiment, a user may select to brush his teeth for example, and choose to select when to apply toothpaste and the quantity of tooth paste. In other embodiments, the user may select to dispense a different oral hygiene solution, such as a tooth whitener or other solutions used to indicate bacterial content as will be further described herein.

Display 314 may be an interactive touch display, to include functionalities of power and dispensing as described herein as one of ordinary skill in the art may appreciate. Display 314 can display a plurality of status and activity options, including, for example, display the operation mode of oral hygiene monitoring device 300, display a series of habits associated with the user, vitals associated with oral hygiene device such as battery life, reminders to the user to use the oral hygiene device 300, and other displays such as instructions, and information associated with the installed cartridge, such as content, levels, and so one as one of ordinary skill in the art would appreciate.

Sensor housing member 316 may include a plurality of sensors, including a battery, motor for further advancing dispensation of the solution out of a cartridge, motion sensors and vibration motors to enhance brushing experience.

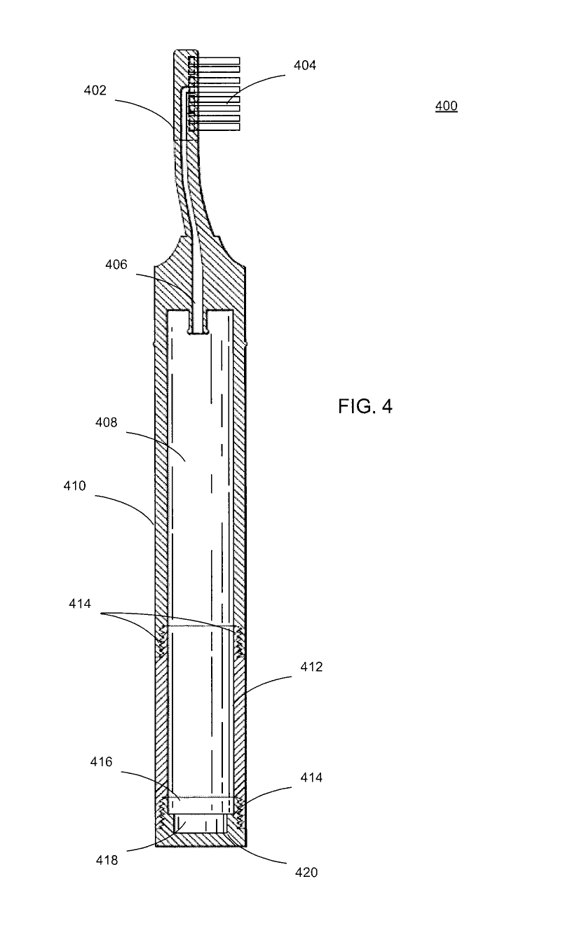

FIG. 4 describes a side view of oral hygiene monitoring device 400 including internal structure according to exemplary aspects of the disclosure. Oral hygiene monitoring device 400 includes head platform 402, bristle assembly 404, fluid conduit 406, cartridge 408, handle member 410, sensor housing member 412, mechanisms 414, motor 416, battery 418 and sensor cap 420.

Head platform 402 may have a conduit housing location that enables the fluid conduit 406 to reach bristle assembly 404. Bristle assembly 404 may include one or more bristle platforms that can have bristles attached to the platforms and the platforms can be assembled to be movable about the head platform 402 as discussed further herein this disclosure. Fluid conduit 406 is made to connect cartridge 408 to bristle assembly 404. Fluid conduit can be made by any flexible plastic material capable of flexing with the rotating movement of head platform 402 and further capable of transforming fluid from the cartridge 408 to the bristle assembly 404. Cartridge assembly 408 is a movable and replaceable cartridge as will be further described hereinafter.

In one embodiment, cartridge 408 may connect to fluid conduit 406 via an interface (not shown) that enables fluid communication as described further hereinafter. Mechanisms 410 are used to enable a user to insert and secure a cartridge 408 within the oral hygiene monitoring device 400. In one example, assemblies 410 may be include spiraling protruding grooves to enable securely screwing on and screwing off each one of handle member 410 together with sensor housing member 412 and sensor cap 420.

Motor 416 may be made to operate in multiple modes, including a mode to create vibrations sufficient to create vibrations at head platform 402 to enhance brushing results. Additionally, motor 416 may be further made to operate to create sufficient vacuum pressure to enable dispensing of fluid from cartridge onto the bristles through fluid conduit 406. Motor 416 may be activated in a plurality of ways, including, for example, activation by a user indicating, via user interface, when to start and stop dispensing fluid. Alternatively, if a motion sensor (not shown) detects user motion, then activation of motor 416 may occur and dispensation of oral hygiene fluid takes place as will be described further hereinafter.

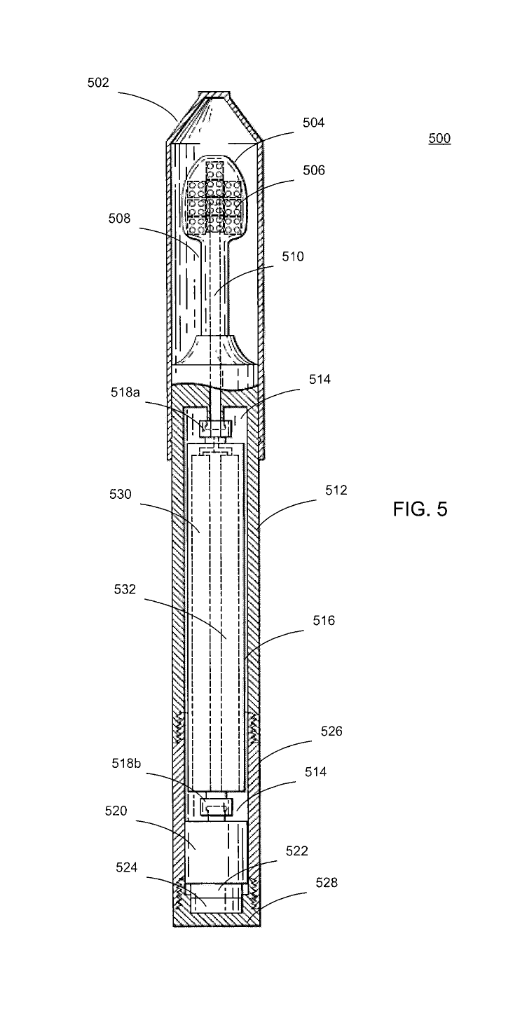

FIG. 5 describes a side view of oral hygiene monitoring device 500 including internal components according to exemplary aspects of the disclosure. Oral hygiene monitoring device 500 includes cap 502, head platform 504, bristle platforms 506, head member 508, fluid conduit 510, handle member 512, cartridge housing 514, cartridge 516, cartridge portals 518a and 518b, motor 520, motion sensors 522, battery 524, sensor housing 526 and sensor cap 528.

In one example, cartridge 516 may be inserted within handle member 512 to reside within allocated space for cartridge housing 514. To securely hold the cartridge, cartridge 516 connects on both ends to cartridge portals 518a and 518b. The connection may be a snap on connection such that each portal includes a protrusion to perform a clip-on operation when pressed against the cartridge.

To insert a cartridge, sensor cap 528 may be removed by unscrewing the cap followed by unscrewing of sensor housing 526. Alternatively, as one of skilled in the art may appreciate, sensor housing 526 includes sensor cap 528 and further includes a series of sensors, such as motor cartridge portal 518b, 520, motion sensors 522 and battery 524. To insert a cartridge in this example, sensor housing may be removed entirely in one operation by unscrewing operation and the cartridge 516 may be inserted in handle member 512. When cartridge 516 is inserted, a snap on connection occurs with cartridge portal 518a. Upon applying a screwing operation to attach sensor housing 526 and all of its components, cartridge portal 518b snaps on to cartridge 516 thus enabling communication between the sensors and the cartridge as will be further discussed hereinafter. Cartridge 516 may be inserted by different means. Oral hygiene monitoring device 500 may include a latch mechanism between handle member 512 and sensor housing member 526 and the connection is made by a hinge like assembly. To insert cartridge 516, sensor housing member 526 may be lifted to an open position while remaining hingedly connected to handle member 512 and placed in closed position when the cartridge is inserted. The snapping operation for cartridge portal 518a may be the same as in the earlier example, e.g., by inserting snapping on cartridge 516 into portal 518a. For cartridge portal 518b, however, the snap on motion may be from a side to side operation.

In one example, cartridge 516 may include a plurality of chambers, such as chamber 530 and chamber 532 disposed within the cartridge 516. Chambers 530 and 532 may include a plurality of oral hygiene fluids, such as tooth paste, mouth wash, disinfectants, fluid conduit cleaning solutions, teeth desensitizing solutions and the like. Chambers 530 and 532 are only examples of a number of chambers within cartridge 516 and cartridge 516 may be set up to house two or more chambers of different solutions as one of ordinary skill in the art may appreciate.

In an operational mode, when cartridge 516 is inserted and connected to cartridge portals 518a and 518b, fluid may be negotiated between the cartridge and cartridge portal 518a and output to fluid conduit 510 to irrigate bristle platforms 506 at predetermined or, alternatively, changing locations. Chambers 530 and 532 may be vacuum sealed or pressure sealed such that cartridge portal 518a may regulate the flow of different fluids from different chambers by opening and closing gates within cartridge chamber 518a. Alternatively, and to ensure constant and measured flow, motor 520 may be activated to induce sufficient pressure through cartridge portal 518b to allow for fluid to flow from any one or more of chambers 530 and 532 into fluid conduit 510 via cartridge portal 518a.

In one example, motor 520 may be activated intentionally by a user, or in the alternative, may be activated by detected brushing motion. A user may decide to, for example to have pre-configured settings to include a specific amount of tooth paste output every time the user uses the brush. As such, either during a detected motion, or by having the user press a predetermined button, such as, for example, button 312 illustrated in FIG. 3A, a predetermined amount of the fluid may be output.

A user may want the freedom to select a brushing technique that is most suitable for the user's comfort level. As such, different configurations of oral hygiene monitoring device 600 may be implementable that best suit the user's desires. In one example, a user may tilt the head member 602 towards different angles to get greater leverage for brushing motion, to improve the reach of the brush for certain areas of the mouth. The tilting of head member 602 may further enable compact design considerations including better fit of the oral hygiene monitoring device within compact locations, such as pockets, travel kits and the like.

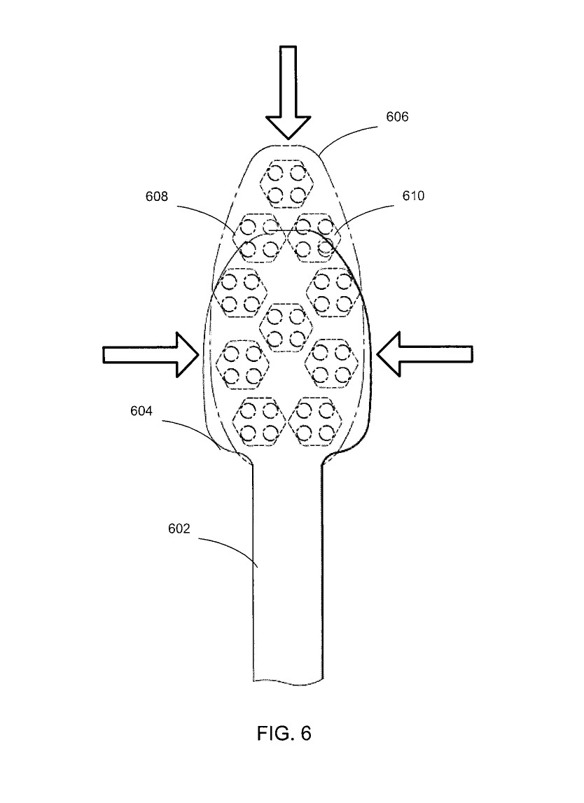

In another example for selecting brushing configurations, FIG. 6 describes a view of a head assembly including a flexible deformable head platform 604 including multiple movable bristle platforms according to exemplary aspects of the disclosure. Head platform 604 sits atop head member 602. When pressure is applied, head platform 604 may expand into a more elongated shape as illustrated be newly formed head platform 606. Bristle plates, as represented by at least bristle plates 608 and 610, for example, can be arranged to be movable within head platform 604 such that they may be arranged and re-arranged in a plurality of potential shapes and organized in a variety of different locations in relation to one another. For example, pressure may be applied one each side of bristle plates 608 and 610 causing head platform 604 to expand into an elongated position 606 and may be further expanded into a further elongated position (not shown). Alternatively, pressure may also be applied at bristle plate location 612 such that elongated head platform 606 may be formed back into original head platform 604. Other manipulations of the head platform are dependent on the user pressure applied at different locations to yield different deformable shapes as required by a user to suite more advanced application for oral hygiene as can be appreciated by one of ordinary skill in the art.

Head platform 604 may be made of any type of deformable material, including, for example, any agile shape-changing polymer material. The polymer material may be able to change shape at preconfigured locations. For example, an air pressure may be applied by motor 522 through each of cartridge portal 518b, a dedicated cartridge chamber, such as cartridge chamber 532 for example, cartridge portal 518a, conduit 518 and onto head platform 504 to expand head platform 504 into a first, second or third predetermined configuration. Conduit 518 may be used, or alternatively, a secondary conduit (not shown) especially dedicated for inflating head platform 504 may be incorporated. A valve system (not shown) may be used to deflate the head platform back into a different position from that of an inflated one.

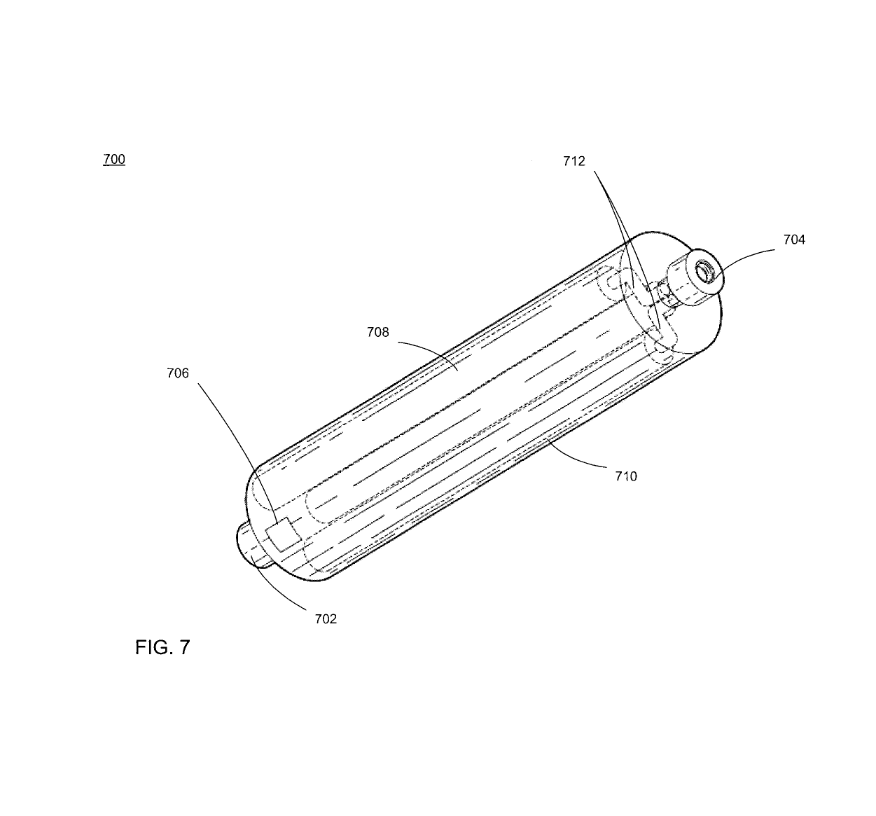

FIG. 7 describes a sample cartridge 700 with internal components and interfaces according to exemplary aspects of the disclosure. Cartridge 700 includes cartridge a lower interface port 702, upper interface port 704, a communication device 706, and chambers 708 and 710. For purposes of illustration, only three chambers are illustrated. However, a plurality of chambers can be configured to reside within cartridge 700 depending on user preferences and purchases. For example, users who only have specific toothpaste requirements may use cartridges with a limited number of chambers, such as 1 or 2 or the like. Alternatively, users wishing to have access to and options to use multiple oral hygiene solutions, such as different types of toothpaste, mouth wash, etc., may use canisters with multiple chambers. Cartridge 700 further includes pipes 712 that connect each chamber to upper interface port 704.

Communication device 706 maybe a near field communication (NFC) patch or a radio frequency identification patch (RFID) used to identify the cartridge and cartridge content. For example, and for the sake of illustration only, communication device 706 is an NFC tag that may be made to include information about cartridge 700 such as the number of chambers, the oral hygiene content for each chamber, levels of the content and the like. When cartridge 700 is within close proximity of oral hygiene monitoring device 500, either nearby the handle member or within handle member 512 or inserted within oral hygiene monitoring device 500, CPU 202 can detect the content of cartridge 700 by reading NFC tag 706. CPU 202 may do so by using an initiator sensor, such as peripheral device 218 that actively generates a radio frequency (RF) field that can power a passive target. A passive target may be NFC tag 706 that can be encoded with cartridge related information and read by peripheral device 218. NFC tag can contain data related to cartridge 700 and be a read-only tag. In other embodiments, NFC tag may be a rewritable tag as one of ordinary skill in the art may appreciate.

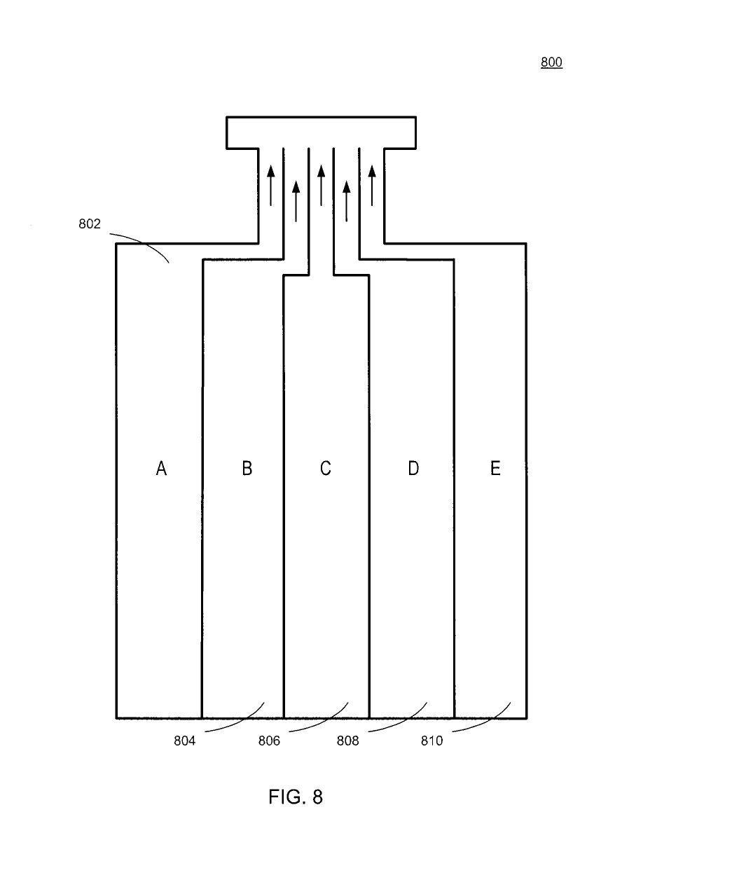

FIG. 8 describes a sample cartridge 800 with possible internal components according to exemplary aspects of the disclosure. Cartridge 800 may include a plurality of chambers, such as chambers 802, 804, 806, 808 and 810. As may be appreciated by one of ordinary skill in the art, cartridge 800 may include one or more chambers of oral hygiene fluid and or compressed air chamber, as applicable for different consumer desires. In one example, cartridge 800 may only include one chamber to hold one type of oral hygiene fluid, such as a type of tooth paste and a second chamber for compressed air for cleaning the oral hygiene fluid conduit 510. Alternatively, the compressed air may but further utilized for other purposes, including but not limited to, expanding deformable head platform 504 into selected and preconfigured shapes.

In another embodiment, cartridge 800 may include chambers for a plurality of different types of oral hygiene solutions, including, but not limited to: different types of tooth paste, different flavors of tooth paste, different manufacturers of tooth paste, mouth wash fluid, compressed water for conduit cleaning, and plaque detecting fluid. Plaque detecting fluid may be utilized so that a user can determine whether or not a visit to a dentist is necessary. Upon selecting the plaque detecting fluid, a user can manipulate the spraying of the fluid by manipulating user interface such as buttons 310 or 312 and/or interactive touch screen 314. Oral hygiene monitoring device 500 may also include a sensor to sense color changes due to interaction of the fluid with plaque dense areas and make automated recommendation as will further be discussed hereinafter.

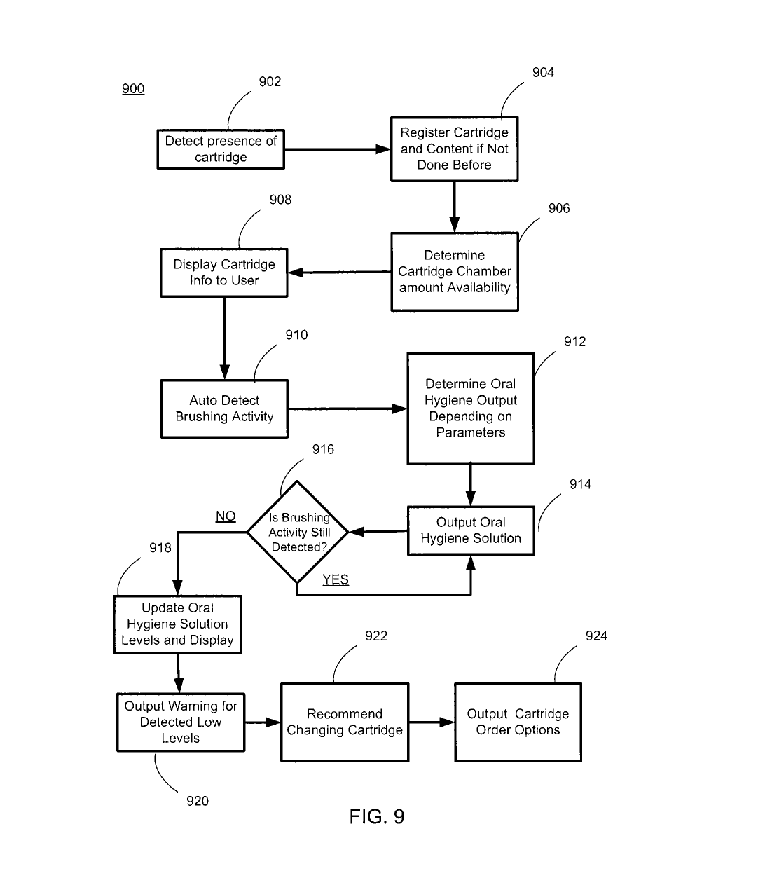

FIG. 9 describes a flow diagram illustration for a method 900 for detecting brushing action and dispensing oral hygiene solution according to exemplary aspects of the disclosure. In one example, method 900 include detecting the presence of a cartridge 902, registering the cartridge and content if not previously done, 904, determine cartridge chamber amount availability 906, display cartridge information to a user 908, automatically detect brushing activity 920, determine oral hygiene output depending on given parameters 912, outputting oral hygiene solution 914, checking if brushing activity is still ongoing 916, updating oral hygiene solution levels and display the levels 918, outputting warning for detected low levels 920, recommending changing cartridge 922 and outputting toothpaste cartridge order options 924.

In one embodiment, detecting the presence of a cartridge 902 may be done by detecting the NFC patch associated with the installed cartridge. Each cartridge includes an NFC patch that includes information related to the content and levels of oral hygiene fluid included within each chamber of the cartridge. After detecting a cartridge, method 900 includes registering the cartridge and its content if not previously done, 904. For example, if a cartridge is newly placed within oral hygiene monitoring device 500, CPU 202 may use peripheral device 218 to excite NFC patch with RF radiation in order to read the information associated with the cartridge. Based on that information, oral hygiene monitoring device 500 may determine the number of cartridge chambers and the oral hygiene content for each chamber and display, 908, that information to the user via display 314 for example.

A user may manually command output of an oral hygiene solution by indicating the need for the output on a user interface, such as buttons 310 or 312 or alternatively, display 314. As previously indicated, display 314 may be a graphical user interface touch screen that can perform both, display of information, as well as receive and communicate user input to oral hygiene monitoring device 500. For example, a user may start brushing his teeth and press button 310 when requiring an output of tooth paste. The user may additionally pre-configure oral hygiene monitoring device 500 to output specific oral hygiene fluids, such as a mix of tooth pastes, a specific flavor of a tooth paste, mouth wash or the like, when a request for fluid output is detected from the user.

Alternatively, oral hygiene monitoring device 500 may automatically detect 910 the brushing activity. Automatic detection may be done in a variety of ways, including, but not limited to, measuring the output of the motion sensors and determining that a brushing motion is under way. When brushing motion is detected, oral hygiene monitoring device 500 can determine 912 the oral hygiene output based on predetermined parameters. For example, the output of the oral hygiene fluid may depend on how long a brushing activity is detected. The longer the activity is detected, the more oral hygiene fluid may be output. In one example, a predetermined amount is output for every detected increment of time. If, for example, the predetermined increment of time is 10 seconds, then oral hygiene is output 6 times for each minute of detected brushing operation. The increments can be counted based on the first output. For example, when a brushing motion is detected, a first output of oral hygiene fluid is provided and the time increment is instituted. After 10 seconds from first output, if a brushing motion is detected again, then a second volume is output and so on.

If brushing activity is still detected at step 916, then continue the incremental output process discussed herein. However, if brushing activity is not detected, then oral hygiene monitoring device 500 updates 918 oral hygiene solution levels and displays those levels to the user. Updating the levels can be done in multiple ways. In one example, initial levels are recorded based on detected NFC information presented by the inserted cartridge initially. Thereafter, output of oral hygiene fluid is measured and amounts are deducted from the initial levels recorded. Amounts can be measured by knowing volume flow for each output at the cartridge portal and determining the detected output for each time interval.

When levels are detected to be low for a particular chamber or for a plurality of chambers in the oral hygiene cartridge, oral hygiene monitoring device 500 may output 920 a warning to inform a user of detected low levels of oral hygiene fluid within the cartridge and recommend, 922, changing the cartridge. Users may order replacement cartridges 924 in accordance with predetermined supplies. For example, cartridges may include, in their registration, how many cartridges are in each order, and oral hygiene monitoring device 500 can track how many cartridges are used. For example, if 8 cartridges come in one package, then the first cartridge will indicate a package of 8 and that when the eighth cartridge is used, the user may be presented with cartridge ordering options.

In some exemplary embodiments, a user may be in transit, on the go, or in need to conduct an oral hygiene activity such as brush his teeth. However, toothpaste may be in short supply, or alternatively, mouth wash may not be available. Alternatively, a user may not want to carry all that supply around. As such, oral hygiene solutions can be presented in compact way embedded within an oral hygiene cartridge as presented herein this disclosure. In attempting to perform an oral hygiene operation, such as brushing teeth, a user may manually choose to dispense the oral hygiene liquid, such as toothpaste, or may allow for oral hygiene device to output the toothpaste based on usage. These settings may be set by a manufacturer, or alternatively, may also be set by the user in accordance with user settings as one of ordinary skill in the art would appreciate.

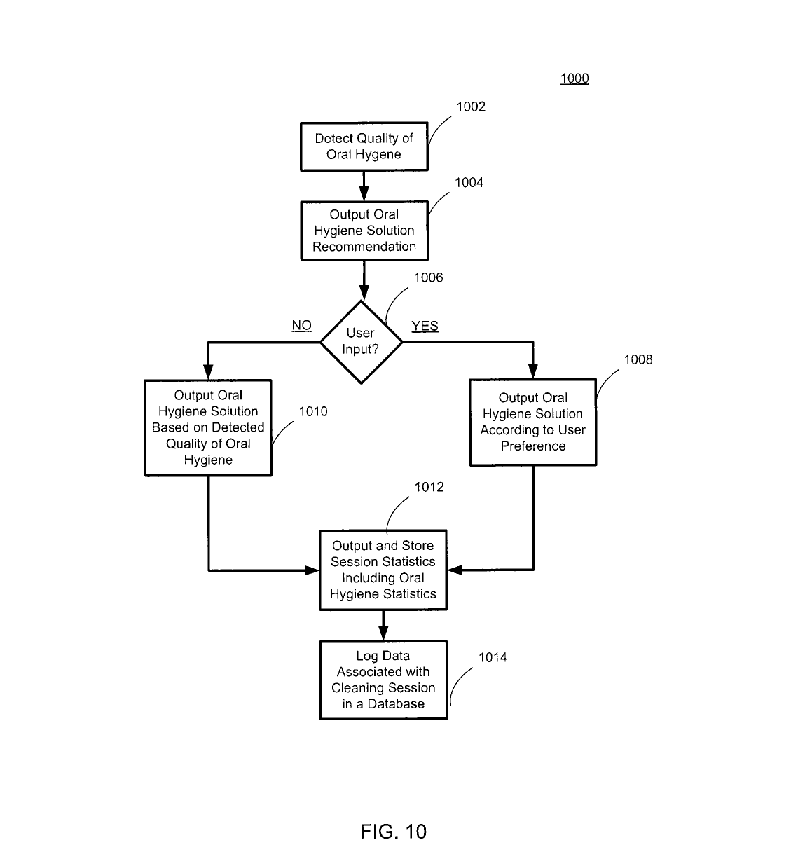

FIG. 10 describes a flow diagram illustration for a method 1000 for measuring, storing and presenting oral hygiene habits according to exemplary aspects of the disclosure. Method 1000 includes detecting quality of oral hygiene 1002, outputting an oral hygiene solution recommendation 1004, detecting user input 1006, if user input detected, the outputting oral hygiene solution according to user preference 1008, otherwise, output oral hygiene solution based on detected quality of oral hygiene 1010, outputting and storing session statistics including oral hygiene statistics 1012 and logging data associated with cleaning session in a database 1014.

Detecting the quality of oral hygiene 1002 includes determining levels of plaque buildup and the presence of other bacteria found in a person's mouth. Detecting quality of oral hygiene 1002 can be done in a verity of ways. One method to detect oral hygiene is to have plaque detecting rinse solution in the cartridge and spray the mouth with the plaque detecting rinse. Plaque detecting rinse will interact with plaque in the mouth and show visible color changes. In a manual setting, a user may determine for himself a plaque level build up based on visual examination and determine an oral hygiene moving forward. This is described in greater detail in step 1008. In an automatic mode, oral hygiene monitoring device 500 would additionally include infrared red (IR) device (not shown) incorporated within head platform 504 that is used to detect changes in color after plaque detecting rinse is applied.

Method 1000 further includes outputting an oral hygiene solution recommendation 1004 after detection is made. In one example, the detection may yield clean results, as such a specific solution is recommended. In other examples, plaque or other bacterial build up may be detected, as such, a more abrasive solution may be recommended. Alternatively, a more teeth sensitive solution may also be recommended. Furthermore, different vibration settings may be set based on detected oral hygiene. For example, if greater plaque buildup is detected, then higher vibration settings may be automatically set by oral hygiene monitoring device 500. When recommendation 1004 is presented, a user input is detected 1006. If a user wishes to manually set oral hygiene solution preferences 1008, then a user may do so at any time. Otherwise, if no user input is detected, method 1000 outputs oral hygiene solution based on the detected quality of oral hygiene. All session statistics associated with detected oral hygiene quality and session statistics are recorded and output 1012 on the display and logged 1014 in a database as discussed further hereinafter.

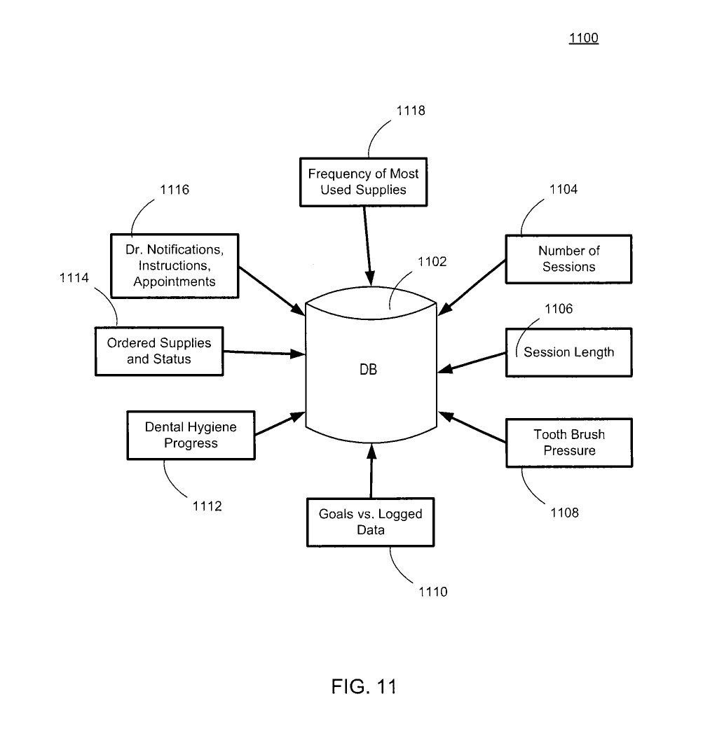

FIG. 11 describes a sample list of measured and stored metrics 1100 associated with oral hygiene activities according to exemplary aspects of the disclosure. Measured and stored metrics are stored in a database 1102. Database 1102 can be housed within an internal memory associated with oral hygiene monitoring device or an external memory associated with an external device, such as a wireless mobile device. Measured and stored metrics include number of sessions 1104, length of each oral hygiene sessions 1106, tooth brush pressure 1108, mapped goals versus logged data 1110, dental hygiene progress 1112, ordered supplies and status 1114 and doctor notifications, instructions and appointments 1116 and frequency of most used supplies 1118.

In one example, the number of sessions is captured and stored in database 1102. Number of sessions 1104 may include daily, weekly, monthly or annual sessions recorded. As such, a display of a number of daily sessions, for example, may be shown. For example, oral hygiene monitoring device may display to a user that the user is averaging a specific number of sessions per selected period, such as 1.5 sessions per day. Or alternatively, 2.5 sessions per day, and so on. Session length 1106 is of relevance in determining user oral hygiene habits. For example, optimal oral hygiene habits may require a predetermined number of minutes for each oral hygiene session, such as 2 minutes. In viewing session length, oral hygiene monitoring device can display to a user, where his status is in comparison to the optimal session length and make recommendations. For example, if a 2 minute brushing session may be ideal, then a 1 minute session average may constitute a warning to be raised to the user's attention. Alternatively, a 4 minute session average may be considered excessive and such a warning may be raised to the user's attention as well.

Another metric that can be measured and stored in database 1102 is toothbrush pressure 1108. This is pressure applied by a user on the teeth or gums using the toothbrush. In one example, pressure sensors may be placed within the head member of the oral hygiene monitoring device to measure the pressure. In alternative embodiments, pressure measurements can be made by measuring rotational pressure at the hinged head member. Detecting tooth brush pressure 1108 can enable a user to determine if they are applying excessive force on their gums, for example, or in the alternative, are barely applying the oral hygiene monitoring device up to its optimal setting to get optimal results.

A user may also set goals for oral hygiene habits. Such goals may include, but are not limited to, increased brushing sessions, session length, dental hygiene progress, and the like. A comparison of input goals versus measured logged data may be presented to a user at any requested time. Alternatively, if a logged data attribute falls below a stated goal, a comparison and alarm may be presented to the user to indicate any potential short coming.

Dental hygiene progress 1112 may also be saved and mapped on display for the user. Dental hygiene progress can be data analysis of goals versus logged data. Additionally, dental hygiene progress 1112 may also include measured quality of dental hygiene over a period of time. For example, if plaque or other oral bacteria is previously detected, this option can illustrate the state change of quality of oral hygiene over a period of time, such as one month or the like.

Supplies can be ordered from and the status of the ordered supplies 1114 can be displayed on oral hygiene monitoring device. Supplies may include cartridges, batteries, head platforms and the like. Additionally doctor's notifications and instruction and appointment status 1116 may further be stored in database 1102 and displayed by the oral hygiene monitoring device. Logged data in database 1102 may be shared with the user's dentist and alarms can be triggered and sent to the dentist to recommend remedial measures. As such, a dentist may send a message notification to the user indicating remedial measures, instructions and reminders. The dental hygiene monitoring device may also include a list of scheduled appointments. The appointments may be related to the dentist or other doctors. Additionally, appointments can be input by a user, or downloaded from a remote server such as a mobile device, or a server associated with the respective doctor's office.

A user may further map out the user's habits to determine frequently used hygiene products 1118. For example, a user may frequently use specific toothpaste or mouth hygiene or a specific flavor. This helps a user to validate user preferences or determine when a change may be recommended.

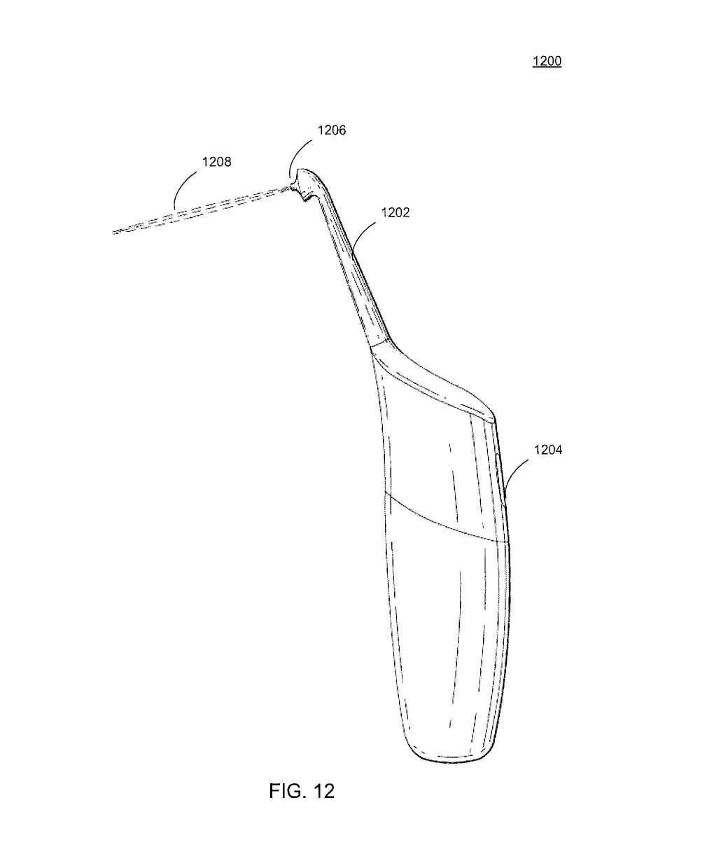

FIG. 12 describes a sample interchangeable head configuration 1200 to output high pressure liquid, steam vapor and/or air according to exemplary aspects of the present disclosure. Head configuration 1200 includes head member 1202, handle member 1204 and head nozzle member 1206. In one example, head nozzle member 1206 connects to a dispensing conduit that is connected to a cartridge placed within handle member 1204 in similar fashion as described in FIGS. 4 and 5.

Depending on a cartridge content and user preferences, a user may select to output a plurality of possible dental solutions 1208 through head nozzle member 1206. For example, a user may select to output high pressure fluid, such as water or mouth wash to remove an object or wash off a previously applied oral hygiene solution. High pressure fluid may also be used for applications where braces are installed so that objects that were not properly removed by brushing application can be further removed using the high pressure fluid.

Steam and high pressure air may also be utilized. For steam, a heating component (not shown) may be installed within handle member 1204 to head liquid exiting the cartridge prior to being output through head nozzle member 1206. The heating component may draw power from the battery assembly, such as battery 418 of FIG. 4, installed within handle member 1204. Steam and high pressure air may also be used to reduce anaerobic bacteria that exist in the mouth, due to bad brushing habits or complex braces assemblies that do not receive the proper attention.

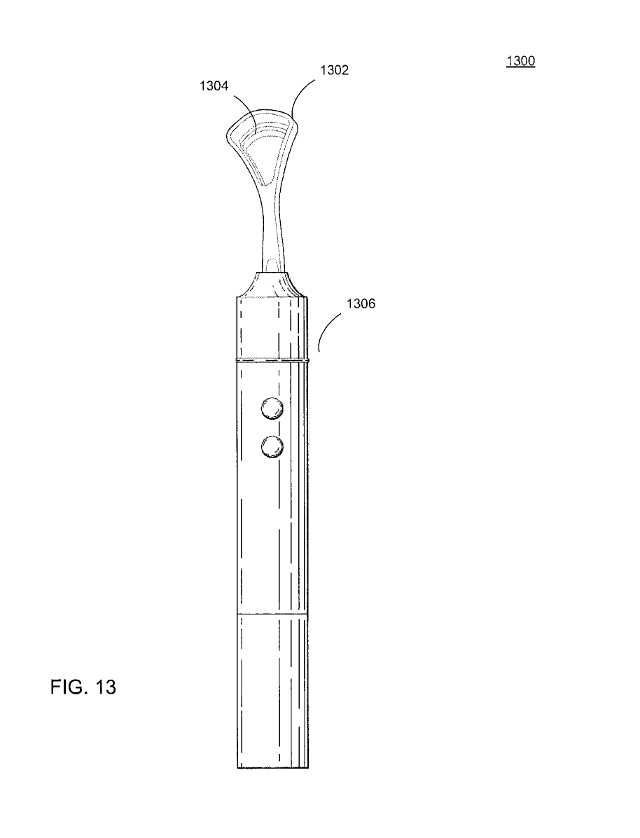

FIG. 13 describes a sample interchangeable head assembly 1300 for aiding in tongue cleaning according to exemplary aspects of the present disclosure. Interchangeable head assembly includes tongue cleaner 1302 that can be inserted and removed from the electronic hygiene monitoring device. Tongue cleaner 1302 may include a series of scrapers 1304 used to remove bacteria and soft plaque in a comfortable and easy way. Tongue cleaner 1302 may be made of any material that is impervious to bacteria such as stainless steel or plastic. Tongue cleaner 1302 may also include a series of bristle assemblies (not shown) designed to further clean the soft tissue of the tongue in addition to the scrapers 1304. In one example, oral hygiene monitoring device 1306 may include motor assembly, such as motor assembly 416 that enables up-down motion or vibration motion for enhanced scraping effect of the tongue cleaner 1302.

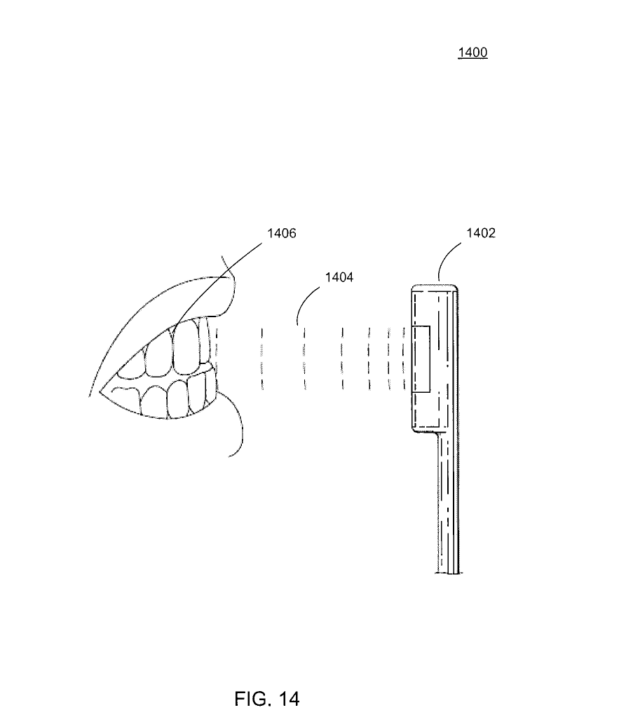

FIG. 14 describes a radiation head configurations 1400 for scanning status of oral hygiene according to exemplary aspects of the present disclosure. Radiation assembly 1400 includes radiation head 1402 that may also be installed within the hygiene monitoring device. Radiation head 1402 may radiate ultrasonic waves or laser waves 1404 on specific areas in the mouth to determine gum inflammation, cavities and the like. Periodontal gum disease is one of the main reasons for tooth loss. Without invasive gum surgery, radiation head 1402 may be used by a user to scan potential gum inflammation or disease.

The non-limiting examples of head configurations are illustrative of possible head configurations that can integrated within the oral hygiene monitoring system described earlier. In one other example, a head with multiple sensor configurations may also be utilized and adapted to communicate results directly with a wireless device and potentially a user's dentist. For example, the head assembly may include a Halimeter that can detect hydrogen sulfide levels in the user's mouth and determine whether a case of halitosis exists or not. A detected case of halitosis, where high levels of hydrogen sulfide are detected in the mouth, would indicate to the user that a case of bad breath is present, and remedial action is recommended. Other sensors may also include, for example, sensors to detect Mercaptan smells, which are smells resulting from odor produced due to foods eaten, such as garlic, onion or the like, despite having a clean mouth. Another sensor that may be integrated within oral hygiene monitoring system includes benzoyl-DL-arginine-naphthylamide (BANA) test such as a BANA strip assembly including a reader integrated within oral hygiene monitoring system.

To expedite diagnostics, a camera assembly may further be integrated within a head assembly and connected to the processor of the oral hygiene monitoring device and may also include wireless connectivity such as that described in FIG. 2. In one example, a user may capture images of areas of the mouth that cannot be reached by a normal camera, such as that found in a camera device or a smart phone device. The captured image may be stored on the oral hygiene monitoring system or may be stored remotely and transmitted to any number of entities designated by the user, such as a user's parent, friend, or dentist.

Thus, the foregoing discussion discloses and describes exemplary embodiments of the present disclosure for clarity. As will be understood by those skilled in the art, the present invention may be embodied in other specific forms without departing from the spirit or essential characteristics thereof and aspects of the exemplary embodiments described herein may be combined differently to form additional embodiments or omitted. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. An oral hygiene system for maintaining and monitoring oral health comprising:
a hollow handle member including an internal chamber configured to house an oral hygiene fluid cartridge;
a head member connected on one end to the handle member by a hinge and configured to rotate at fixed angular increments within a range of 0 to 180 degrees between the head member and the handle member;
a head platform secured to an end of the head member opposite from the handle member and including at least one bristle platform;
a fluid conduit disposed within the head member and connected between the head platform and a fluid negotiating portal within the internal chamber of the hollow handle member;
a fluid cartridge including at least one oral hygiene fluid chamber and a negotiating neck configured to attach to the negotiating portal; and
circuitry configured to
detect a brushing motion,
identify at least one of the at least one oral hygiene fluid within the oral hygiene fluid chamber and identify a quantity of the at least one of the at least one oral hygiene fluid, and
regulate a predetermined output quantity of the oral hygiene fluid to the head platform via the fluid conduit based on the detected brushing motion.

2. The oral hygiene system of claim 1, wherein the head platform is an elastically deformable head platform comprising at least two bristle platforms that are configured to change disposition with regards to one another within the elastically deformable head platform.

3. The oral hygiene system of claim 2, wherein each of the at least two bristle platforms includes at least one irrigation nozzle operatively connected to the oral hygiene fluid conduit, and the irrigation nozzle is configured to output oral hygiene fluid received from the oral hygiene fluid conduit onto a bristle surface of each of the at least two bristle platforms.

4. The oral hygiene system of claim 3, wherein the irrigation nozzle is a bristle within each of the at least two bristle platforms.

5. The oral hygiene system of claim 2, wherein the elastically deformable head platform has dimensions that are modifiable in longitudinal and latitudinal directions.

6. The oral hygiene system of claim 1, wherein the fluid cartridge includes one or more oral hygiene fluid chambers and at least one of the chambers contains fluid to clean the oral hygiene fluid conduit of residue resultant from prior uses.

7. The oral hygiene system of claim 6, wherein the one or more oral hygiene fluid chambers are vacuum sealed or pressure sealed.

8. The oral hygiene system of claim 6, wherein when the fluid cartridge is connected to the fluid negotiating portal, a flow of oral hygiene fluid is regulated by a fluid regulator configured to negotiate fluid flow from the oral hygiene fluid chambers into the fluid conduit.

9. The oral hygiene system of claim 1, wherein the oral hygiene fluid cartridge further includes a near field communication (NFC) tag that is configured to automatically identify the type and content of the fluid cartridge when the fluid cartridge is within the oral hygiene system.

10. The oral hygiene system of claim 9, wherein the oral hygiene fluid is toothpaste.

11. The oral hygiene system of claim 10, wherein different types of toothpaste are housed in within different fluid chambers.

12. The oral hygiene system of claim 11, where the different types of toothpaste include one or more combination of different flavored toothpaste, different texture tooth paste, mouth wash, fluid conduit cleaner, and tooth whitener.

13. The oral hygiene system of claim 12, wherein the circuitry further comprising:
a memory configured to store usage preferences and prior usage history, wherein the circuitry is further configured to regulate an output quantity from different oral hygiene fluid chambers based on stored usage preferences or prior usage history.

14. The oral hygiene system of claim 13, wherein the circuitry is further configured to measure a quality of dental hygiene and automatically regulate an output of oral hygiene fluid from at least one chamber of the oral hygiene fluid cartridge based on the measured quality of dental hygiene.

15. The oral hygiene system of claim 14, wherein the measured quality of dental hygiene is related to plaque buildup.

16. A method of monitoring and maintaining dental hygiene comprising:
automatically detecting brushing activity using motion detection circuitry;
determining toothpaste quantity based on tracked average history and detected motion;
measuring a quality of dental hygiene;
displaying a toothpaste solution based on the measured quality of dental hygiene detecting toothpaste levels in a toothpaste cartridge;
outputting warning to change toothpaste cartridge when levels falls below a threshold; and
displaying toothpaste cartridge order options.

17. The method of claim 16, further comprising:
irrigating an oral hygiene fluid conduit with oral hygiene fluid from an oral hygiene fluid cartridge in response to a detected brushing activity; and
irrigating the oral hygiene fluid conduit with cleaning fluid from the oral hygiene fluid cartridge after the detected brushing activity ends.

18. A non-transitory computer readable medium having computer-readable instructions thereon which when executed by a computer cause the computer to perform a method according to claim 16.
